(19) United States
(12) Patent Application Publication
(10) Pub. No.: US 2010/0029476 A1
(43) Pub. Date: Feb. 4, 2010

(54) MAGNESIUM BUTYLISOPHTHALATE AS A POROUS METAL ORGANIC FRAMEWORK

(75) Inventors: Natalia Trukhan, Ludwigshafen (DE); Ulrich Mueller, Neustadt (DE); Markus Schubert, Ludwigshafen (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/445,322

(22) PCT Filed: Nov. 5, 2007
(86) PCT No.: PCT/EP07/61863

§ 371 Date: Apr. 13, 2009

(30) Foreign Application Priority Data
Nov. 6, 2006 (EP) 06123525.5

(51) Int. Cl. C07C 55/02; B01J 20/22

(52) U.S. Cl. 502/401; 562/590

(57) ABSTRACT

The present invention relates to a porous metal organic framework formed by Mg²⁺ ions to which 5-tert-butylisophthalate ions are coordinated to form a framework structure. The invention further provides a process for preparing it and its use, for example for the storage, separation or controlled release of a substance such as a gas or gas mixture.

The present invention relates to a porous metal organic framework, its preparation and use.

Porous metal organic frameworks are known from the prior art. They are notable for, in particular, their porosity and can be employed in applications comparable to those of inorganic zeolites.

Metal organic frameworks usually comprise an at least bidentate organic compound coordinated to a metal ion; this organic compound together with the metal ion forms the skeleton of the metal organic framework.

Appropriate choice of metal and/or organic compound makes it possible to optimize the metal organic framework for the desired field of application. Here, for example, the choice of organic compound can influence the pore distribution. In addition, the metal can make a contribution in adsorption processes.

There is therefore a continual need to provide specific metal organic frameworks which have, in particular, extraordinary properties which are attributable to the choice of metal and of organic compound.

As an interesting metal, mention may be made of magnesium since, owing to strong coordinate bonds, it is possible to start out from a comparatively narrow-pored framework and because magnesium is a comparatively unproblematical metal both physiologically and ecologically.

M. Dinca et al., J. Am. Chem. Soc. 127 (2005), 9376-9377, describe magnesium 2,6-naphthalenedicarboxylate as microporous solid having a coordinate structure. This framework displays a structure analogous to the corresponding zinc-based metal organic framework. In the examination of the material, the authors have found that it has a high hydrogen adsorption enthalpy and displays selective adsorption of hydrogen or oxygen over nitrogen or carbon monoxide.

WO-A 2005/049892 describes the electrochemical preparation of magnesium terephthalate in the presence of diethyl maleate as porous metal organic framework. The framework obtained in this way is likewise comparable to the corresponding zinc-based metal organic framework in terms of its structure.

Despite the promising results obtained using the magnesium-based metal organic framework known from the prior art, there continues to be a need for alternative framework structures which can be achieved by appropriate choice of metal and organic compound.

It is therefore an object of the present invention to provide such a magnesium-based metal organic framework.

This object is achieved by a porous metal organic framework formed by Mg²⁺ ions to which 5-tert-butylisophthalate ions are coordinated to form a framework structure.

It has surprisingly been found that the framework of the invention has a surprisingly high specific surface area compared to the analogous magnesium isophthalate and is suitable, in particular, for separations of gases which may comprise gaseous water.

The framework of the invention is formed by Mg²⁺ ions and 5-tert-butylisophthalate acid (5-^{t}butyl-1,3-benzenedicarboxylic acid) or its anionic forms.

The metal organic framework of the invention can be in powder form or be present as agglomerates.

The porous metal organic framework of the invention can be used as such in powder form or is converted into a shaped body. Accordingly, it is a further aspect of the present invention that the porous metal organic framework of the invention is present as part of a shaped body.

The production of shaped bodies from metal organic frameworks is described, for example, in WO-A 03/102000.

Preferred processes for producing shaped bodies are extrusion or tableting. In the production of shaped bodies, the framework can comprise further materials such as binders, lubricants or other additives which are added during production. It is likewise conceivable for the framework to comprise further constituents such as absorbents such as activated carbon or the like.

The possible geometries of these shaped bodies are subject to essentially no restrictions. Examples are, inter alia, pellets such as disk-shaped pellets, pills, spheres, granules, extrudates such as rod extrudates, honeycombs, grids and hollow bodies.

All suitable processes are in principle possible for producing these shaped bodies. In particular, the following processes are preferred:
Kneading/pan milling of the framework either alone or together with at least one binder and/or at least one pasting agent and/or at least one template compound to give a mixture; shaping of the resulting mixture by means of at least one suitable method, for example extrusion; optionally washing and/or drying and/or calcination of the extrudate; optionally finishing.
Tableting together with at least one binder and/or other auxiliary.
Application of the framework to at least one optionally porous support material. The material obtained can then be processed further by the method described above to give a shaped body.
Application of the framework to at least one optionally porous substrate.

Kneading/pan milling and shaping can be carried out by any suitable method, as described, for example, in Ullmanns Enzyklopädie der Technischen Chemie, 4th edition, Volume 2, p. 313 ff. (1972).

For example, the kneading/pan milling and/or shaping can be carried out by means of a piston press, roll press in the presence or absence of at least one binder material, compounding, pelletization, tableting, extrusion, coextrusion, foaming, spinning, coating, granulation, preferably spray granulation, spraying, spray drying or a combination of two or more of these methods.

Very particular preference is given to producing pellets and/or tablets.

The kneading and/or shaping can be carried out at elevated temperatures, for example in the range from room temperature to 300° C., and/or at elevated pressure, for example in the range from atmospheric pressure to a few hundred bar, and/or in a protective gas atmosphere, for example in the presence of at least one noble gas, nitrogen or a mixture of two or more thereof.

The kneading and/or shaping is, according to a further embodiment, carried out with addition of at least one binder, with the binder used being able in principle to be any chemical compound which ensures the desired viscosity for kneading and/or shaping the composition. Accordingly, binders can, for the purposes of the present invention, be either viscosity-increasing or viscosity-reducing compounds.

Preferred binders are, for example, inter alia aluminum oxide or binders comprising aluminum oxide as described, for example, in WO 94/29408, silicon dioxide as described, for example, in EP 0 592 050 A1, mixtures of silicon dioxide and aluminum oxide as described, for example, in WO 94/13584, clay minerals as described, for example, in JP 03-037156 A, for example montmorillonite, kaolin, bentonite, hallosite, dickite, nacrite and anauxite, alkoxysilanes as described, for example, in EP 0 102 544 B1, for example tetraalkoxysilanes such as tetramethoxysilane, tetraethoxysilane, tetrapropoxysilane, tetrabutoxysilane, and, for example, trialkoxysilanes such as trimethoxysilane, triethoxysilane, tripropoxysilane, tributoxysilane, alkoxytitanates, for example tetraalkoxytitanates such as tetramethoxytitanate, tetraethoxytitanate, tetrapropoxytitanate, tetrabutoxytitanate, and, for example, trialkoxytitanates such as trimethoxytitanate, triethoxytitanate, tripropoxytitanate, tributoxytitanate, alkoxyzirconates, for example tetraalkoxyzirconates such as tetramethoxyzirconate, tetraethoxyzirconate, tetrapropoxyzirconate, tetrabutoxyzirconate, and, for example, trialkoxyzirconates such as trimethoxyzirconate, triethoxyzirconate, tripropoxyzirconate, tributoxyzirconate, silica sols, amphiphilic substances and/or graphites.

As viscosity-increasing compound, it is also possible, for example, to use, if appropriate in addition to the abovementioned compounds, an organic compound and/or a hydrophilic polymer such as cellulose or a cellulose derivative such as methylcellulose and/or a polyacrylate and/or a polymethacrylate and/or a polyvinyl alcohol and/or a polyvinylpyrrolidone and/or a polyisobutene and/or a polytetrahydrofuran and/or a polyethylene oxide.

As pasting agent, preference is given to using, inter alia, water or at least one alcohol, for example a monoalcohol having from 1 to 4 carbon atoms, e.g. methanol, ethanol, n-propanol, isopropanol, 1-butanol, 2-butanol, 2-methyl-1-propanol or 2-methyl-2-propanol, or a mixture of water and at least one of the alcohols mentioned or a polyhydric alcohol such as a glycol, preferably a water-miscible polyhydric alcohol, either alone or as a mixture with water and/or at least one of the monohydric alcohols mentioned.

Further additives which can be used for kneading and/or shaping are, inter alia, amines or amine derivatives such as tetraalkylammonium compounds or amino alcohols and carbonate-comprising compounds such as calcium carbonate. Such further additives are described, for instance, in EP 0 389 041 A1, EP 0 200 260 A1 or WO 95/19222.

The order of addition of the additives such as template compound, binder, pasting agent, viscosity-increasing substance in shaping and kneading is in principle not critical.

In a further preferred embodiment, the shaped body obtained by kneading and/or shaping is subjected to at least one drying operation which is generally carried out at a temperature in the range from 25 to 500° C., preferably in the range from 50 to 500° C. and particularly preferably in the range from 100 to 500° C. It is likewise possible to carry out drying under reduced pressure or under a protective gas atmosphere or by spray drying.

In a particularly preferred embodiment, at least one of the compounds added as additives is at least partly removed from the shaped body during this drying operation.

The metal organic framework of the invention comprises pores, in particular micropores and/or mesopores. Micropores are defined as pores having a diameter of 2 nm or less and mesopores are defined by a diameter in the range from 2 to 50 nm. The presence of micropores and/or mesopores can be checked with the aid of sorption measurements, with these measurements determining the uptake capacity of the metal organic frameworks for nitrogen at 77 kelvin in accordance with DIN 66131 and/or DIN 66134.

The specific surface area, calculated according to the Langmuir model (DIN 66131, 66134), of an MOF in powder form is preferably more than 5 m²/g, more preferably above 10 m²/g, more preferably more than 50 m²/g, even more preferably more than 100 m²/g, even more preferably more than 200 m²/g and particularly preferably more than 300 m²/g.

Shaped bodies comprising metal organic frameworks can have a lower specific surface area; however, this is preferably more than 10 m²/g, more preferably more than 50 m²/g, even more preferably more than 100 m²/g, in particular more than 200 m²/g.

The pore size of the metal organic framework of the invention is preferably from 0.2 nm to 30 nm, particularly preferably in the range from 0.3 nm to 3 nm, based on the crystalline material.

However, larger pores whose size distribution can vary also occur in a shaped body of the metal organic framework of the invention. However, preference is given to more than 50% of the total pore volume, in particular more than 75%, being formed by pores having a pore diameter of up to 1000 nm. However, a major part of the pore volume is preferably formed by pores in two diameter ranges. It is therefore further preferred that more than 25% of the total pore volume, in particular more than 50% of the total pore volume, is formed by pores in a diameter range from 100 nm to 800 nm and that more than 15% of the total pore volume, in particular more than 25% of the total pore volume, is formed by pores in a diameter range up to 10 nm. The pore distribution can be determined by means of mercury porosimetry.

The present invention further provides a process for preparing a porous metal organic framework according to the invention, which comprises the step
reaction of a magnesium compound with 5-tert-butylisophthalic acid or a salt thereof. 5-tert-Butylisophthalic acid serves as organic component of the porous metal organic framework of the invention and can be reacted with a magnesium compound. It is likewise possible to use derivatives of 5-tert-butylisophthalic acid. Thus, it is conceivable, for example, for 5-tert-butylisophthalic acid to be used in the form of its salt. The salt, in which 5-tert-butylisophthalic acid is present as fully or partially deprotonated anion, can have any suitable cation. Such cations can, for example, be monovalent or divalent metal ions. Examples are, in particular, sodium and potassium salts. Cations of ammonium compounds can likewise be used. Mention may here be made of, in particular, ammonium itself and also alkylammonium cations.

The magnesium compound can be produced by anodic oxidation of metallic magnesium. In such a case, the porous metal organic framework of the invention is prepared by an electrochemical route. Processes for the electrochemical preparation of porous metal organic frameworks are described in WO-A 2005/049892. The porous metal organic framework of the invention, too, can be prepared in this way.

In the electrochemical preparation of the porous metal organic framework of the invention, cathodic redeposition of the magnesium ion is preferably at least partially prevented by at least one of the following measures:

(i) use of an electrolyte which promotes the cathodic formation of hydrogen;
(ii) addition of at least one compound which leads to cathodic depolarization;
(iii) use of a cathode having a suitable hydrogen overvoltage.

The process can be carried out in an undivided electrolysis cell. Particularly suitable cells are gap cells or stacked plate cells. These can be connected in a bipolar fashion. Suitable reaction media are, for example, methanol, ethanol, dimethylformamide, diethylformamide and mixtures of two or more of these solvents.

Furthermore, an electrolyte salt or a plurality of electrolyte salts can be present in the reaction medium. Here, the electrolyte salt can have a quaternary ammonium as cation component and an alkoxysulfate as anion component. The total solids content should be in the range of greater than or equal to 0.5% by weight.

The reaction in the process of the invention for preparing the metal organic framework of the invention can be carried out in the classical way. Here, the magnesium compound is typically a magnesium salt.

The magnesium salt can be present in the form of an alkoxide, acetonate, halide, sulfite, salt of an organic or inorganic, oxygen-comprising acid or a mixture thereof.

An alkoxide is, for example, a methoxide, ethoxide, n-propoxide, i-propoxide, n-butoxide, i-butoxide, t-butoxide or phenoxide.

An acetonate is, for example, acetylacetonate. A halide is, for example, chloride, bromide or Iodide.

An organic, oxygen-comprising acid is, for example, formic acid, acetic acid, propionic acid or another alkylmonocarboxylic acid.

An inorganic, oxygen-comprising acid is, for example, sulfuric acid, sulfurous acid, phosphoric acid or nitric acid.

Here, the magnesium occurs as Mg²⁺ cation.

Further preferred magnesium compounds are inorganic magnesium salts such as magnesium chloride, magnesium bromide, magnesium hydrogensulfate, magnesium dihydrogenphosphate, magnesium monohydrogenphosphate, magnesium nitrate.

The magnesium compound can, if appropriate, comprise water of hydration.

The reaction in the process of the invention for preparing the porous metal organic framework of the invention can be carried out in an aqueous medium. Here, hydrothermal conditions or solvothermal conditions in general can be used. For the purposes of the present invention, the term “thermal” refers to a preparative process in which the reaction to form the porous metal organic framework of the invention is carried out in a pressure vessel in such a way that this is closed during the reaction and an elevated temperature is applied so that a pressure builds up in the reaction medium in the pressure vessel as a result of the vapor pressure of solvent present.

However, the reaction is preferably not carried out in an aqueous medium and likewise not under solvothermal conditions.

The reaction in the process of the invention is preferably carried out in the presence of a nonaqueous solvent.

The reaction is preferably carried out at a pressure of not more than 2 bar (absolute). However, the pressure is preferably not more than 1230 mbar (absolute). In particular, the reaction preferably takes place at atmospheric pressure. However, slightly superatmospheric or subatmospheric pressures can also occur due to the apparatus. For the purposes of the present invention, the term “atmospheric pressure” therefore means the pressure range given by the actual atmospheric pressure ±150 mbar.

The reaction can be carried out at room temperature. However, it preferably takes place at temperatures above room temperature. The temperature is preferably more than 100° C. Furthermore, the temperature is preferably not more than 180° C. and more preferably not more than 150° C.

The above-described metal organic frameworks are typically prepared in water as solvent with addition of a further base. The latter serves, in particular, to make a polybasic carboxylic acid used as at least bidentate organic compound readily soluble in water. As a result of the preferred use of the nonaqueous organic solvent, it is not necessary to use such a base. Nonetheless, the solvent for the process of the invention can be selected so that it has a basic reaction, but this is not absolutely necessary for carrying out the process of the invention.

It is likewise possible to use a base. However, preference is given to using no additional base.

It is also advantageous for the reaction to be able to take place with stirring, which is also advantageous in a scale-up.

The nonaqueous organic solvent is preferably a C₁₋₆-alkanol, dimethyl sulfoxide (DMSO), N,N-dimethylformamide (DMF), N,N-diethylformamide (DEF), acetonitrile, toluene, dioxane, benzene, chlorobenzene, methyl ethyl ketone (MEK), pyridine, tetrahydrofuran (THF), ethyl acetate, optionally halogenated C₁₋₂₀₀-alkane, sulfolane, glycol, N-methylpyrrolidone (NMP), gamma-butyrolactone, alicyclic alcohols such as cyclohexanol, ketones such as acetone or acetylacetone, cyclic ketones such as cyclohexanone, sulfolene or a mixture thereof.

A C₁₋₆-alkanol is an alcohol having from 1 to 6 carbon atoms. Examples are methanol, ethanol, n-propanol, i-propanol, n-butanol, i-butanol, t-butanol, pentanol, hexanol and mixtures thereof.

An optionally halogenated C₁₋₂₀₀-alkane is an alkane having from 1 to 200 carbon atoms in which one or more up to all hydrogen atoms may be replaced by halogen, preferably chlorine or fluorine, in particular chlorine. Examples are chloroform, dichloromethane, tetrachloromethane, dichloroethane, hexane, heptane, octane and mixtures thereof.

Preferred solvents are DMF, DEF and NMP. Particular preference is given to DMF.

The term “nonaqueous” preferably refers to a solvent which has a maximum water content of 10% by weight, more preferably 5% by weight, even more preferably 1% by weight, still more preferably 0.1% by weight, particularly preferably 0.01% by weight, based on the total weight of the solvent.

The maximum water content during the reaction is preferably 10% by weight, more preferably 5% by weight and even more preferably 1% by weight.

The term “solvent” encompasses pure solvents and mixtures of various solvents.

Furthermore, the process step of the reaction of the at least one metal compound with the at least one at least bidentate organic compound is preferably followed by a calcination step. The temperature set here is typically above 250° C., preferably from 300 to 400° C.

The at least bidentate organic compound present in the pores can be removed by means of the calcination step.

In addition or as an alternative thereto, the removal of the at least bidentate organic compound (ligand) from the pores of the porous metal organic framework can be effected by treatment of the framework formed with a nonaqueous solvent. Here, the ligand is removed in the manner of an “extraction process” and, if appropriate, replaced in the framework by a solvent molecule. This mild method is particularly useful when the ligand is a high-boiling compound.

The treatment is preferably carried out for at least 30 minutes and can typically be carried out for up to 2 days. This can occur at room temperature or elevated temperature. It is preferably carried out at elevated temperature, for example at least 40° C., preferably 60° C. Further preference is given to the extraction taking place at the boiling point of the solvent used (under reflux).

The treatment can be carried out in a simple vessel by slurrying and stirring the framework. It is also possible to use extraction apparatuses such as Soxhlet apparatuses, in particular industrial extraction apparatuses.

Suitable solvents are those mentioned above, i.e., for example, C₁₋₆-alkanol, dimethyl sulfoxide (DMSO), N,N-dimethylformamide (DMF), N,N-diethylformamide (DEF), acetonitrile, toluene, dioxane, benzene, chlorobenzene, methyl ethyl ketone (MEK), pyridine, tetrahydrofuran (THF), ethyl acetate, optionally halogenated C₁₋₂₀₀-alkane, sulfolane, glycol, N-methylpyrrolidone (NMP), gamma-butyrolactone, alicyclic alcohols such as cyclohexanol, ketones such as acetone or acetylacetone, cyclic ketones such as cyclohexanone or mixtures thereof.

Preference is given to methanol, ethanol, propanol, acetone, MEK and mixtures thereof.

A very particularly preferred extractant is methanol.

The solvent used for extraction can be identical to or different from that used for the reaction of the at least one metal compound with the at least one at least bidentate organic compound. In particular, it is not absolutely necessary but is preferred that the solvent used in the “extraction” is water-free.

The present invention further provides for the use of a porous metal organic framework according to the invention for the uptake of at least one substance for the purposes of its storage, separation, controlled release or chemical reaction.

The at least one substance is preferably a gas or gas mixture, with the gas or gas mixture preferably comprising gaseous water.

In this way, it is possible, in particular to separate off gases or gas mixtures in the presence of water without the water interfering in the separation by being separated off instead of the gas or gas mixture.

The present invention further provides for the use of a porous metal organic framework according to the invention as support or precursor material for preparing a corresponding metal oxide (MgO).

Storage processes using metal organic frameworks in general are described in WO-A 2005/003622, WO-A 2003/064030, WO-A 2005/049484 and in WO-A 2006/089908 and DE-A 10 2005 012 087. The processes described there can also be used for the metal organic framework of the invention.

Separation or purification processes using metal organic frameworks in general are described in EP 1 674 555 and in DE-A 10 2005 000938 and DE-A 10 2005 022 844. The processes described there can also be used for the metal organic framework of the invention.

If the porous metal organic framework of the invention is used for storage, this is preferably effected in the temperature range from −200° C. to +80° C. Greater preference is given to the temperature range from −40° C. to +80° C.

The at least one substance can be a gas or a liquid. The substance is preferably a gas.

For the purposes of the present invention, the terms “gas” and “liquid” are used in the interests of simplicity, but gas mixtures and liquid mixtures or liquid solutions are likewise encompassed by the term “gas” or “liquid”.

Preferred gases are hydrogen, natural gas, town gas, hydrocarbons, in particular methane, ethane, ethene, acetylene, propane, n-butane and i-butane, carbon monoxide, carbon dioxide, nitrogen oxides, oxygen, sulfur oxides, halogens, halogenated hydrocarbons, NF₃, SF₆, ammonia, boranes, phosphanes, hydrogen sulfide, amines, formaldehyde, noble gases, in particular helium, neon, argon, krypton and xenon.

However, the at least one substance can also be a liquid. Examples of such liquids are disinfectants, inorganic or organic solvents, fuels, in particular gasoline or diesel, hydraulic fluids, radiator fluids, brake fluids or an oil, in particular machine oil. Furthermore, the liquid can also be a halogenated aliphatic or aromatic, cyclic or acyclic hydrocarbon or a mixture thereof. In particular, the liquid can be acetone, acetonitrile, aniline, anisole, benzene, benzonitrile, bromobenzene, butanol, tert-butanol, quinoline, chlorobenzene, chloroform, cyclohexane, diethylene glycol, diethyl ether, dimethylacetamide, dimethylformamide, dimethyl sulfoxide, dioxane, glacial acetic acid, acetic anhydride, ethyl acetate, ethanol, ethylene carbonate, ethylene dichloride, ethylene glycol, ethylene glycol dimethyl ether, formamide, hexane, isopropanol, methanol, methoxypropanol, 3-methyl-1-butanol, methylene chloride, methyl ethyl ketone, N-methylformamide, N-methylpyrrolidone, nitrobenzene, nitromethane, piperidine, propanol, propylene carbonate, pyridine, carbon disulfide, sulfolane, tetrachloroethene, carbon tetrachloride, tetrahydrofuran, toluene, 1,1,1-trichloroethane, trichloroethylene, triethylamine, triethylene glycol, triglyme, water or a mixture thereof.

The at least one substance can also be an odorous substance.

The odorous substance is preferably a volatile organic or inorganic compound which comprises at least one of the elements nitrogen, phosphorus, oxygen, sulfur, fluorine, chlorine, bromine or iodine or is an unsaturated or aromatic hydrocarbon or a saturated or unsaturated aldehyde or a ketone. More preferred elements are nitrogen, oxygen, phosphorus, sulfur, chlorine, bromine; and particular preference is given to nitrogen, oxygen, phosphorus and sulfur.

In particular, the odorous substance is ammonia, hydrogen sulfide, sulfur oxides, nitrogen oxides, ozone, cyclic or acyclic amines, thiols, thioethers and also aldehydes, ketones, esters, ethers, acids or alcohols. Particular preference is given to ammonia, hydrogen sulfide, organic acids (preferably acetic acid, propionic acid, butyric acid, isobutyric acid, valeric acid, isovaleric acid, caproic acid, heptanoic acid, lauric acid, pelargonic acid) and cyclic or acyclic hydrocarbons which comprise nitrogen or sulfur and also saturated or unsaturated aldehydes such as hexanal, heptanal, octanal, nonanal, decanal, octenal or nonenal and in particular volatile aldehydes such as butyraldehyde, propionaldehyde, acetaldehyde and formaldehyde and also fuels such as gasoline, diesel (constituents).

The odorous substances can also be fragrances which are used, for example, for producing perfumes. Examples of fragrances or oils which release such fragrances are: essential oils, basil oil, geranium oil, mint oil, cananga oil, cardamom oil, lavender oil, peppermint oil, nutmeg oil, camomile oil, eucalyptus oil, rosemary oil, lemon oil, lime oil, orange oil, bergamot oil, muscatel sage oil, coriander oil, cypress oil, 1,1-dimethoxy-2-phenylethane, 2,4-dimethyl-4-phenyltetrahydrofuran, dimethyltetrahydrobenzaldehyde, 2,6-dimethyl-7-octen-2-ol, 1,2-diethoxy-3,7-dimethyl-2,6-octadiene, phenylacetaldehyde, rose oxide, ethyl 2-methylpentanoate, 1-(2,6,6-trimethyl-1,3-cyclohexadien-1-yl)-2-buten-1-one, ethyl vanillin, 2,6-dimethyl-2-octenol, 3,7-dimethyl-2-octenol, tert-butylcyclohexyl acetate, anisyl acetate, allyl cyclohexyloxyacetate, ethyllinalool, eugenol, coumarin, ethyl acetoacetate, 4-phenyl-2,4,6-trimethyl-1,3-dioxane, 4-methylene-3,5,6,6-tetramethyl-2-heptanone, ethyl tetrahydrosafranate, geranyl nitrile, cis-3-hexen-1-ol, cis-3-hexenyl acetate, cis-3-hexenyl methyl carbonate, 2,6-dimethyl-5-hepten-1-al, 4-(tricyclo[5.2.1.0]decylidene)-8-butanal, 5-(2,2,3-trimethyl-3-cyclopentenyl)-3-methylpentan-2-ol, p-tert-butyl-alpha-methylhydrocinnamaldehyde, ethyl[5.2.1.0]tricyclodecanecarboxylate, geraniol, citronellol, citral, linalool, linalylacetate, ionone, phenylethanol and mixtures thereof.

For the purposes of the present invention, a volatile odorous substance preferably has a boiling point or boiling range below 300° C. The odorous substance is more preferably a readily volatile compound or mixture. The odorous substance particularly preferably has a boiling point or boiling range below 250° C., more preferably below 230° C., particularly preferably below 200° C.

Preference is likewise given to odorous substances which have a high volatility. The vapor pressure can be employed as a measure of the volatility. For the purposes of the present invention, a volatile odorous substance preferably has a vapor pressure of more than 0.001 kPa (20° C.). The odorous substance is more preferably a readily volatile compound or mixture. The odorous substance particularly preferably has a vapor pressure of more than 0.01 kPa (20° C.), more preferably a vapor pressure of more than 0.05 kPa (20° C.). Particular preference is given to the odorous substances having a vapor pressure of more than 0.1 kPa (20° C.).

In addition, it has been found to be advantageous that the porous metal organic framework of the invention can be used for preparing a magnesium oxide. Here, the metal organic framework of the invention is heated to above its complete decomposition temperature.

The heating can be effected by methods known to those skilled in the art. Heating is typically carried out in a furnace which is suitable for this purpose, for example a muffle furnace. When using a furnace, it is also advantageous for facilities which enable heating to be carried out in the presence of a suitable atmosphere to be present. For this purpose, a feed line for an appropriate gas or gas mixture can be appropriately installed in or on the furnace so that the furnace chamber comprising the porous metal organic framework can be flooded with the appropriate gas or gas mixture.

The porous metal organic framework is heated to the temperature necessary to convert the metal organic framework into the corresponding metal oxide. It is therefore heated to above the complete decomposition temperature of the metal organic framework.

For the purposes of the present invention, the “complete decomposition temperature” is the temperature at which the porous metal organic framework starts to be converted into the corresponding metal oxide. However, it is likewise possible for the metal organic framework to be converted into the metal oxide via intermediates. For example, a carbonate could have been formed before formation of the metal oxide. In such a case, the “complete decomposition temperature” is the temperature necessary to convert the last intermediate in each case into the metal oxide.

The determination of the complete decomposition temperature can be carried out by methods known to those skilled in the art. For example, this temperature can be determined thermogravimetrically, with confirmation of the formation of the corresponding metal oxide likewise being able to be carried out by accompanying analysis.

The complete decomposition temperature which is necessary to produce the corresponding metal oxide from the porous metal organic framework is typically in the range from 250° C. to 1000° C. The complete decomposition temperature is more preferably in the range from 350° C. to 800° C. The complete decomposition temperature is very particularly preferably in the range from 450° C. to 650° C.

The heating of the porous metal organic framework therefore takes place in the presence of an oxidizing atmosphere comprising an oxygen-supplying constituent. In this way, it can be ensured that sufficient oxygen for converting the porous metal organic framework into the corresponding metal oxide is available. This can also, in particular, contribute to the abovementioned intermediates being “leapfrogged”. Such oxidizing atmospheres can be obtained by means of appropriate oxygen-supplying gases or gas mixtures. As simplest and most preferred gas mixture, mention may here be made of air which normally comprises a sufficiently high proportion of molecular oxygen. If appropriate, the air used can be enriched with further oxygen. Finally, it is of course likewise possible for pure oxygen to be used as oxidizing atmosphere. In addition, other gases or gas mixtures which are, for example, enriched with molecular oxygen can also be used. Here, particular preference is given to inert gases. Thus, helium, argon, nitrogen or mixtures thereof in each case enriched with oxygen can be used as gas mixtures for producing an oxidizing atmosphere during heating of the porous metal organic framework.

The porous metal organic framework of the invention can be exposed to an oxidizing atmosphere in such a way that the atmosphere is not altered during heating. The gas or gas mixture surrounding the porous metal organic framework is thus not replaced, so that the concentration of the oxygen-supplying constituent of the atmosphere decreases during heating.

In addition, it is possible to keep the concentration of the oxygen-supplying constituent in the atmosphere approximately constant during heating by further introduction of at least this constituent.

However, preference is given to the concentration of the oxygen-supplying constituent being increased during heating. This can be effected, for example, by the atmosphere being replaced by a gas or gas mixture having a higher proportion of oxygen-supplying constituent. This can be achieved, in particular, by introducing oxygen into the atmosphere after commencement of heating until finally a pure oxygen atmosphere is present. The increase can be carried out stepwise or continuously.

Examples of chemical reactions which can take place in the presence of the metal organic framework of the invention are the alkoxylation of monools and polyols. The way in which such alkoxylations can be carried out is described in WO-A 03/035717 and WO-A 2005/03069. The porous metal organic framework of the invention can likewise be used for epoxidation and the preparation of polyalkylene carbonates and hydrogen peroxide. Such reactions are described in WO-A 03/101975, WO-A 2004/037895 and US-A 2004/081611.

### EXAMPLE 1

A mixture of 9.5 g of magnesium nitrate hexahydrate, 2.78 g of 5-tert-butylisophthalic acid and 283 g of diethylformamide (DEF) is stirred at 130° C. under an N₂ atmosphere in a 500 ml flask for 24 hours. The mixture is then cooled to room temperature and the product which has precipitated is filtered off, washed four times with 50 ml each time of acetone and subsequently blown dry by means of N₂ in a wash bottle provided with a frit for 2 days.

This gives 2.60 g of a dry framework.

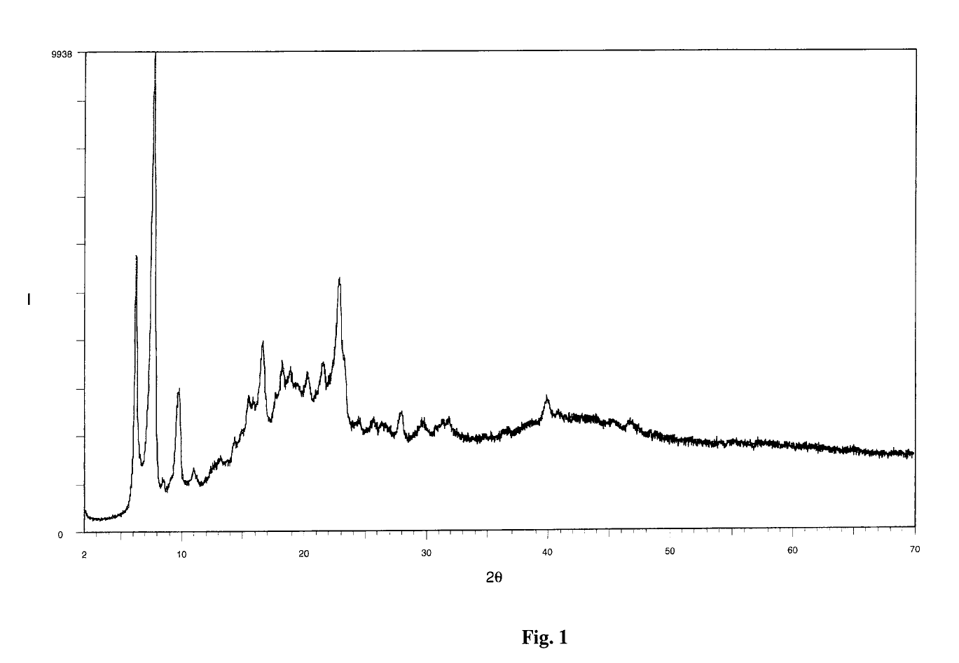

FIG. 1 shows the associated X-ray diffraction pattern (XRD), with I indicating the intensity (Lin(Counts)) and 2Θ describing the 2-theta scale.

The specific surface area determined by the Langmuir method is 326 m²/g. Thermal decomposition takes place at about 470° C.

### COMPARATIVE EXAMPLE 2

A mixture of 11.0 g of magnesium nitrate hexahydrate, 5.00 g of 1,3-benzenedicarboxylic acid (isophthalic acid) and diethylformamide (DEF) is stirred at 130° C. in a 200 ml steel autoclave having a Teflon inside coating for 24 hours. The mixture is then cooled to room temperature and the product which has precipitated is filtered off, washed with N,N-dimethylformamide (2×30 ml) and chloroform (2×30 ml) and subsequently dried in air.

This gives 7.80 g of a dry framework.

No specific surface area could be determined by the Langmuir method.

### EXAMPLE 3

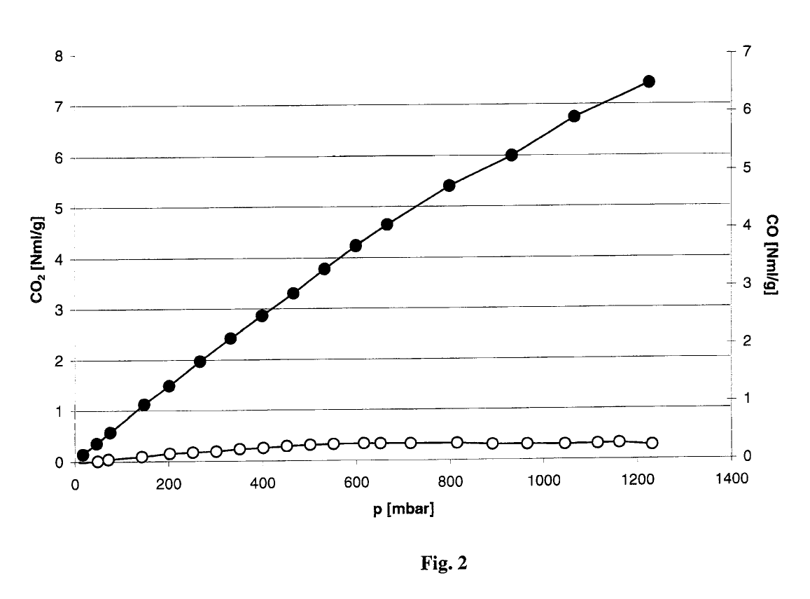

FIG. 2 shows the adsorption isotherme of the framework material of example 1 for CO₂ and CO 313K. The upper curve represents CO₂, the lower CO. The curves demonstrates that a CO₂/CO separation is possible.

1: A porous metal organic framework formed by Mg²⁺ ions to which 5-tert-butylisophthalate ions are coordinated to form a framework structure.

2: The framework according to claim 1 present as part of a shaped body.

3: A process for preparing a metal organic framework according to claim 1, which comprises
reacting magnesium compound with 5-tert-butylisophthalic acid or a salt thereof.

4: The process according to claim 3, wherein the magnesium compound is produced by anodic oxidation of metallic magnesium.

5: The process according to claim 3, wherein the magnesium compound is a magnesium salt.

6-8. (canceled)

9: A method for storing, separating or controlled releasing of at least one substance comprising up-taking the at least one substance by a metal organic framework material of claim 1.

10: The method according to claim 9, wherein the substance is a gas or gas mixture.

11. The method according to claim 10, wherein the gas or gas mixture comprises gaseous water.
